(19) United States
(12) Patent Application Publication
(10) Pub. No.: US 2021/0071770 A1
(43) Pub. Date: Mar. 11, 2021

(54) Leash Valve Apparatus

(71) Applicant: Willow Innovations, Inc., Mountain View, CA (US)

(72) Inventors: Brian Mason, Lexington, MA (US); Taylor Walsh, Philadelphia, PA (US); Jenny Teng, Belmont, CA (US); Erica Keenan, San Francisco, CA (US); John Chang, Los Altos, CA (US)

(21) Appl. No.: 17/094,900

(22) Filed: Nov. 11, 2020

Related U.S. Application Data
(63) Continuation of application No. PCT/US19/31643, filed on May 10, 2019.
(60) Provisional application No. 62/670,730, filed on May 12, 2018.
(60) Provisional application No. 62/789,013, filed on Jan. 7, 2019.

(51) Int. Cl. F16K 15/14; A61M 1/06

(52) U.S. Cl. CPC F16K 15/147; A61M 2205/14; F16K 15/148; A61M 1/062

(57) ABSTRACT

A valve apparatus or assembly that includes a leash. In one aspect, the valve apparatus or assembly includes a loop or hoop structure that attaches to a component of a breast pump system.

### BACKGROUND OF THE DISCLOSURE

The present disclosure relates to valves generally, and in particular to valve apparatus or assemblies for breast pump fluid containers.

As more women become aware that breastfeeding is the best source of nutrition for a baby, and also offers health benefits to the nursing mother, the need is increasing for breast pump solutions that are user-friendly, quiet, discrete and versatile for use by a nursing mother in various situations. This is particularly true for the working mother, who is away from the home for eight to ten hours or more and needs to pump breast milk in order to have it available for her baby, but it is also a requirement for many other situations where the mother is away from the privacy of the home for an extended period, such as during shopping, going out to dinner or other activities.

Although a variety of breast pumps are available, a number are awkward and cumbersome, requiring many parts and assemblies and being difficult to transport. Hand pump varieties that are manually driven are onerous to use and can be inconvenient to use. Some powered breast pumps require an AC power source to plug into during use. Some systems are battery driven, but draw down the battery power fairly rapidly as the motorized pump continuously operates to maintain suction during the milk extraction process. Certain other of the breast pumps available are lacking in convenient connecting and disconnection from storage containers. The storage containers can be difficult to package, install, remove and store thereby creating a barrier to effective use. There is thus a continuing need for conveniently usable and effective approach to attachable and replaceable valve apparatus and assemblies for portable wearable breast pump.

The present disclosure addresses these and other needs.

### SUMMARY OF THE DISCLOSURE

Briefly and in general terms, the present disclosure is directed towards a leash valve apparatus or assembly. In a preferred embodiment, the leash valve is configured to be replaceably attached to a breast pump system component.

In one aspect, the valve apparatus includes a valve portion and a leash portion extending from the valve portion. The valve portion includes a base and a generally cylindrical portion extending from the base. Configured within the cylindrical portion is a valve. Extending laterally from the base is the leash portion, the leash portion terminating with a loop or hoop.

Various types of valves can be incorporated into the valve apparatus. In a preferred embodiment, the valve is an umbrella valve. The valve can be integrally formed in the valve apparatus or can be a component part inserted or attached to the base. In alternative approaches, the valve can define a duckbill valve or a double tailed umbrella valve, or other means or structure that accomplishes a seal. Such structures can also be integrally formed or a component part of an assembly.

In one or more embodiments, the valve assembly includes a separate air path configured to facilitate detection by the system of the presence of a container assembly. The valve assembly is arranged such that the air path is occluded when a container assembly is connected to the breast pump system.

These and other features of the disclosure will become apparent to those persons skilled in the art upon reading the details of the systems and methods as more fully described below.

### BRIEF DESCRIPTION OF THE DRAWINGS

FIGS. 1A-G are perspective and various angle views, depicting a valve apparatus of the present disclosure.

FIGS. 2A-B are front and side cross-sectional views, depicting the valve apparatus of FIGS. 1A-G.

FIG. 3 is a perspective view, depicting a breast pump system.

FIG. 4A is a perspective view, depicting a portion of an interior of the breast pump system of FIG. 3.

FIGS. 4B-C are perspective views, depicting placing a container assembly within a breast pump system.

FIG. 4D is a front view, depicting a portion of a container assembly with a fitment.

FIG. 5 is perspective view, depicting a valve apparatus attached to a breast pump system component.

FIG. 6 is a perspective view, depicting an alternative approach to a valve apparatus.

FIG. 7 is a side view, depicting an alternative valve structure.

FIGS. 8A-G are various views, depicting yet another approach to a valve apparatus.

FIGS. 9A-F are various views, depicting approaches to a valve apparatus including a second air path for detection purposes.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “a sensor” includes a plurality of such sensors and reference to “the system” includes reference to one or more systems and equivalents thereof known to those skilled in the art, and so forth.

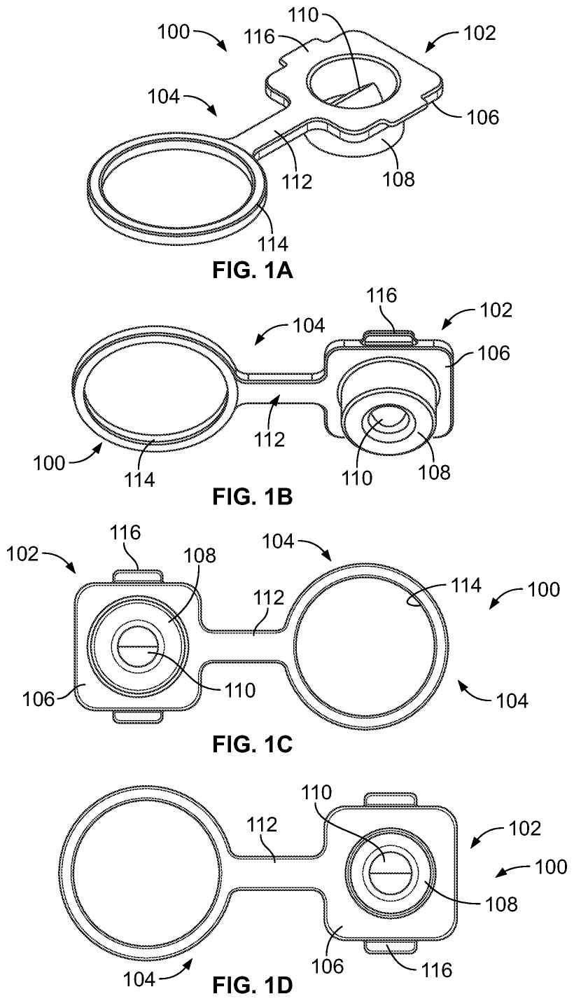

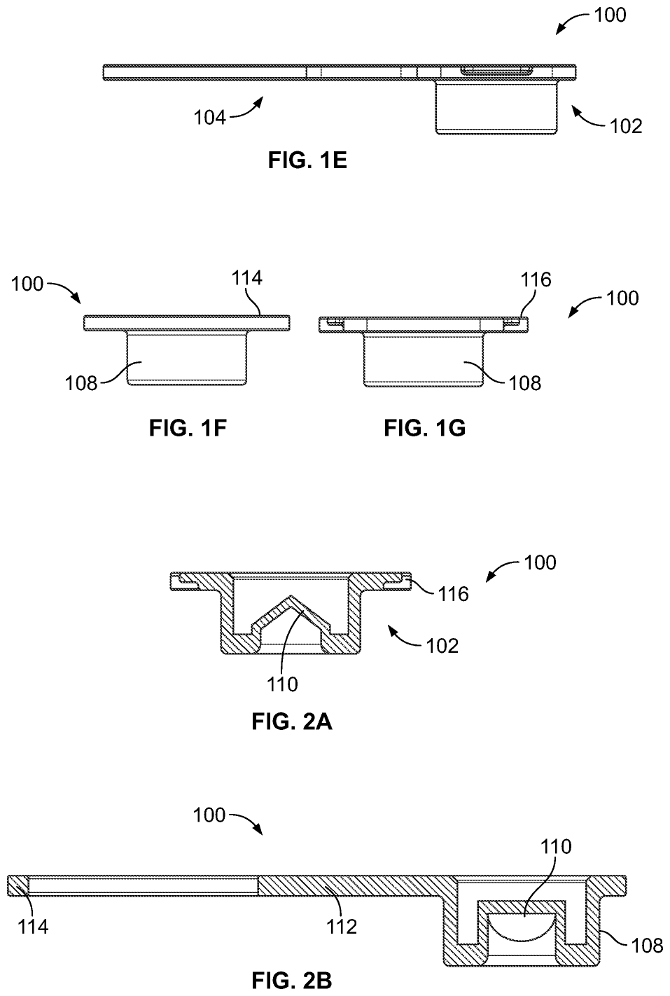

With reference to FIGS. 1A-G, there is shown valve apparatus or assembly 100. In a preferred embodiment, the valve apparatus or assembly include a valve portion 102 and a leash portion 104 extending from the valve portion 102. The valve portion includes a generally rectangular or square base 106 and a generally cylindrical portion 108 extending from the base 106. Configured within the cylindrical portion 108 is a duckbill valve 110. Extending laterally from the base 106 is the leash 112, the leash 112 terminating with a loop or hoop 114. Also extending from opposite sides of the base 106 are a pair of wings 116.

With reference to FIGS. 2A-B, there are shown cross-sectional views of the valve apparatus or assembly 100. In particular, the structure of the cylindrical portion 108 and duckbill valve 110 of the valve portion 102 of the valve apparatus or assembly 100 is depicted.

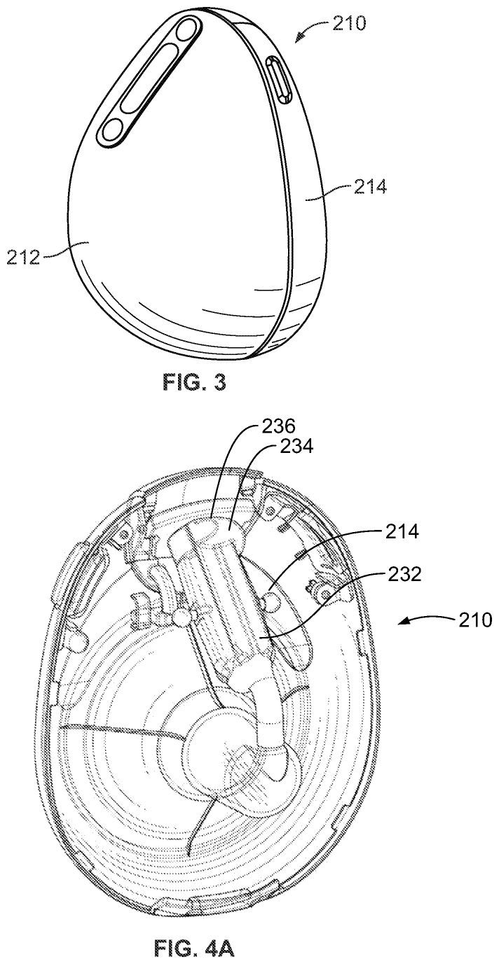

Turning to FIG. 3, there is shown a perspective view of a breast pump system 210 according to an embodiment of the present disclosure. The housing or outer shell 212 of system 10 can be shaped and configured to be contoured to the breast of a user and to thus provide a more natural appearance when under the clothing of the user. As can be appreciated from the figures, the system can define a natural breast profile. The natural breast profile is contemplated to fit comfortably and conveniently into a bra of a user and to present a natural look. Extending from the base are curved surfaces having asymmetric patterns. Moreover, like natural breasts, the profile of the device or system is contemplated to define one or more asymmetric curves and off-center inertial centers. Various natural breast shapes can be provided to choose from to the tastes and needs of a user. An opposite side of the pump system 210 is configured with a flange 214 which is sized and shaped to engage a breast of a user. The flange 214 is contoured to comfortably fit against a wide range of user's bodies and to provide structure for sealingly engaging with breast tissue.

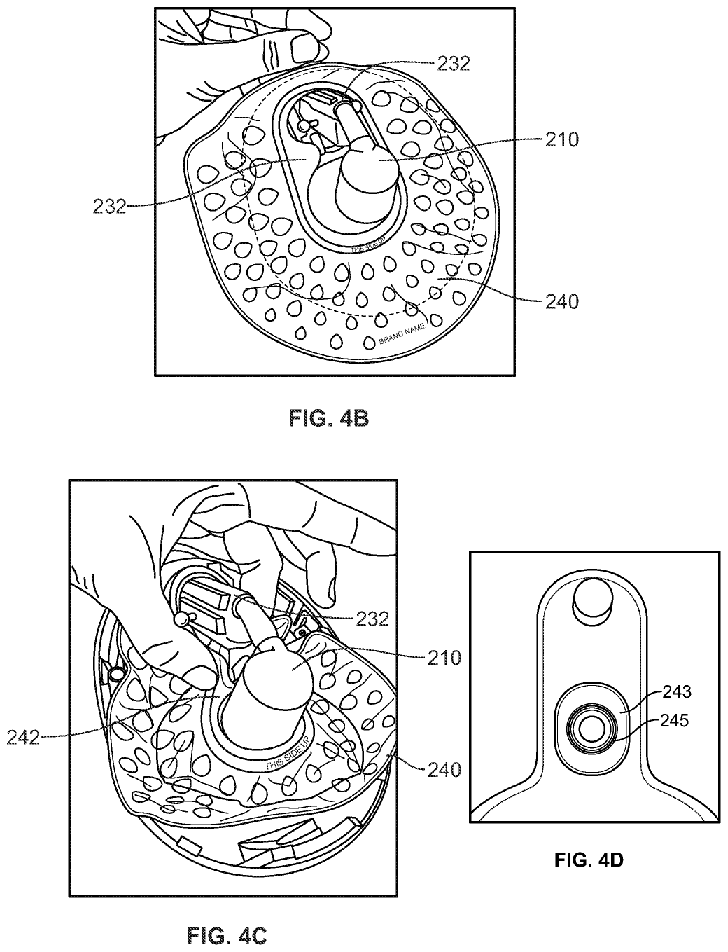

FIG. 4A shows certain features of an interior of the breast pump system 210. In particular, an interior of the flange 214 is shown as is a flextube or conduit 232 configured within the interior of the flange. Various steps in loading a container assembly 240 into the system is shown in FIGS. 4B-C. In a first step (FIG. 4B), the flange 214 is removed from engagement with the remainder of the system 210. Attached to the flange is the flex conduit or tube 232. A central portion 242 of the container assembly 240 is placed over a central projection of the flange 214 and the flex conduit or tube 232. Next, the user can pinch the container assembly and configure it under the flex conduit or tube 232 followed by tucking the container 240 into the flange 214. The opening to the container 240 is fluidly connected to the flex conduit or tube 232 such as via the pump outlet. The opening to the container 240 is configured with structure that mates with the presently disclosed valve apparatus (See FIG. 4D). In one approach, a fitment 243 is configured at the opening to the container 240, the fitment 243 including an opening 245 that mates with the valve apparatus.

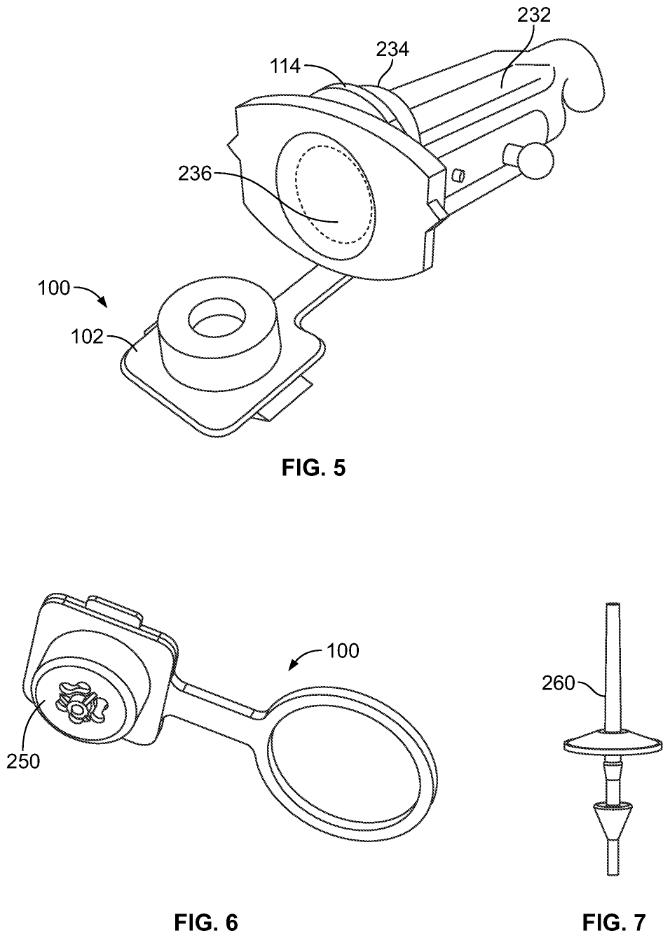

FIG. 5, shows the flextube 232 removed from the breast pump system 210, and with the loop or hoop 114 configured, in a replaceable manner about a portion 234 of the flextube 232. The cylindrical portion 108 of the valve apparatus or assembly 100 is sized and shaped to be securely engaged, and removed from, an opening 236 to the flextube 232. As so configured or assembled, the valve 100 provides a sealed engagement with the flextube 232 so that a vacuum can be created prior to pushing fluid through the valve 100 and into a fluid container (not shown) also attached to the valve 100. This approach removes valve structure from the fluid container thus reducing the cost and simplifying manufacture of the fluid container. It also permits the user to remove air from the fluid container before storage.

Various types of valves can be incorporated into the valve apparatus. In a preferred embodiment, the valve is a duckbill valve. The valve can be integrally formed in the valve apparatus or can be a component part inserted or attached to the base. In alternative approaches (See FIGS. 6 and 7), the valve can define an umbrella valve 250 or a double tailed umbrella valve 260, or other means or structure that accomplishes a seal. Such structures can also be integrally formed or a component part of an assembly.

In one aspect, the valve apparatus or assembly 100 can be made from flexible material such as TPE or silicone. In embodiments including an umbrella valve 250, the body of the valve 100 can be made from both flexible material and/or a stiff material (PP, PE, LDPE), and the umbrella valve portion 250, 252 can be formed from silicone.

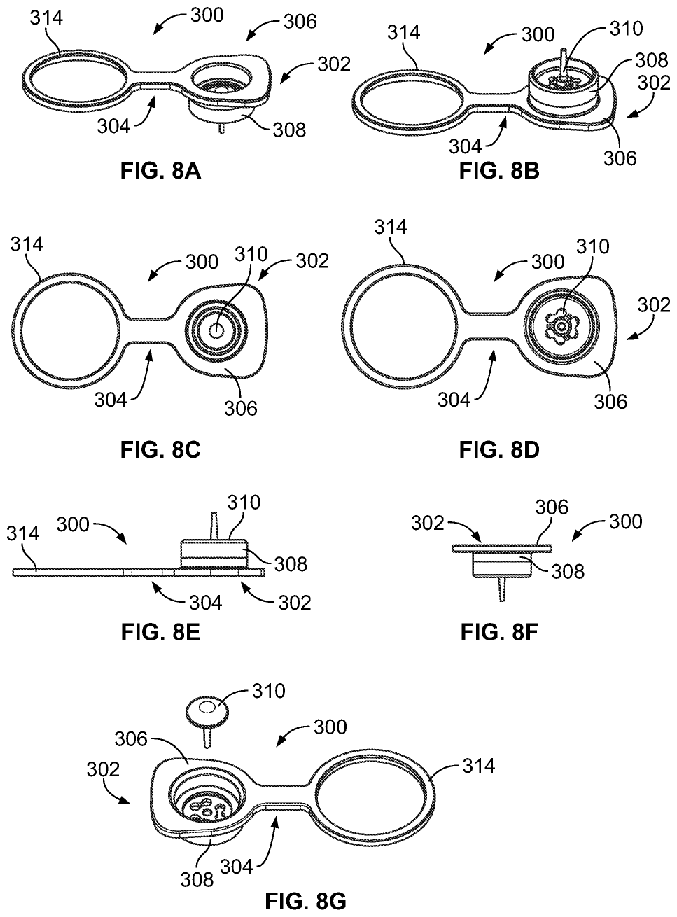

With reference to FIGS. 8A-G, another embodiment of a valve apparatus 300 is shown. The valve apparatus or assembly 300 includes a valve portion 302 and a leash portion 304 extending from the valve portion 302. The valve portion includes a generally rectangular or square base 106 and a generally cylindrical portion 308 extending from the base 306. Configured within the cylindrical portion 308 is an umbrella valve 310. Extending laterally from the base 306 is the leash 312, the leash 312 terminating with a loop or hoop 314.

As with the previous embodiment, the loop or hoop 314 is configured in a replaceable manner about a portion of the flextube. The cylindrical portion 308 of the valve apparatus or assembly 300 is sized and shaped to be securely engaged, and removed from, an opening to the flextube. The valve 310 thus provides a sealed engagement with the flextube so that a vacuum can be created prior to pushing fluid through the valve 100 and into a fluid container also attached to the valve 300. As stated, this approach removes valve structure from the fluid container thus reducing the cost and simplifying manufacture of the fluid container, and also permits the user to remove air from the fluid container before storage.

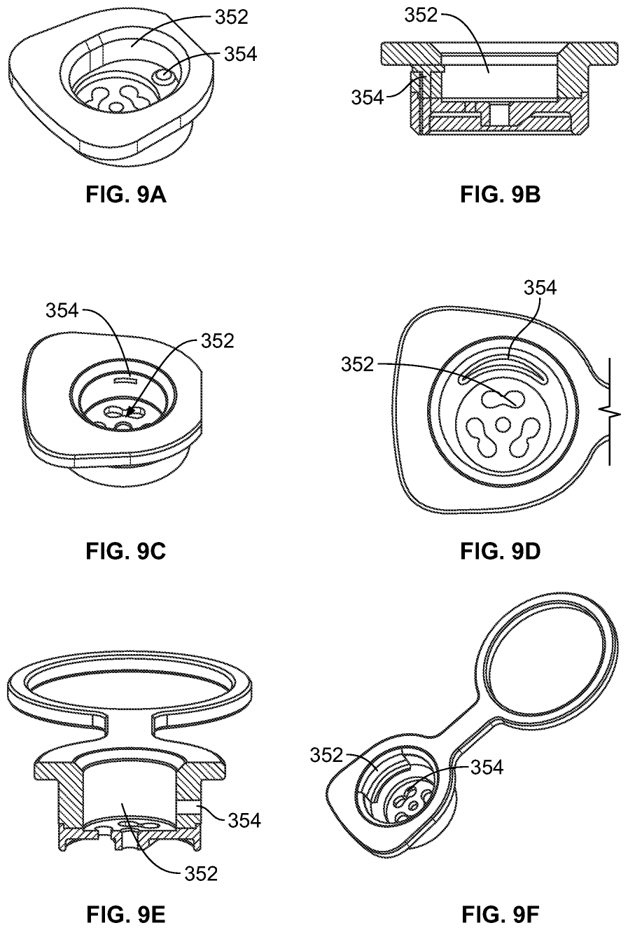

Turning now to FIGS. 9A-F, there are shown valve apparatus including various approaches to facilitating detecting the presence of a container assembly. In a first approach (FIG. 9A), the valve assembly includes a container fitment receiving portion 352 that is non-cylindrical unlike the generally cylindrical container fitment receiving portions previously presented. Here, the receiving portion 352 is generally oval thus providing space for an opening 354 configured to be occluded when a fitment of a container assembly (See FIG. 4D) is attached to the valve assembly. The opening 354 can be a circular hole or a slot. As shown in FIGS. 9B-F, various other shapes of holes, openings or slots 354 can be configured within walls defining a fitment receiving portion 352 to also provide structure configured to facilitate detection of the presence of a container assembly. For example, the hole 354 can define a T-channel (FIG. 9B-C) through the bottom and the side of the fitment receiving portion 352 and the valve structure can be offset in the fitment receiving portion 352 to create more space for the hole formed in the bottom or side of the fitment receiving portion 352 (FIG. 9D). Moreover, the opening 354 can be in a side wall and/or can be keyed such that an inserted container fitment completes the fitment receiving portion (FIGS. 9E and 9F).

Accordingly, various approaches to valve apparatus or assemblies are presented. The disclosed embodiments are configured to provide an effective approach to sealing components of a breast pump system and to facilitate ease of use and manufacture.

While the present disclosure has been described with reference to the specific embodiments thereof, it should be understood by those skilled in the art that various changes may be made, and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A valve apparatus for a breast pump system, comprising:
a valve portion;
a leash portion extending from the valve portion.

2. The valve apparatus of claim 1, wherein the valve portion includes a generally rectangular or square base and a generally cylindrical portion extending from the base.

3. The valve apparatus of claim 2, further comprising a cylindrical portion extending from the base and a valve configured in the cylindrical portion.

4. The valve apparatus of claim 2, further comprising a leash extending laterally from the base.

5. The valve apparatus of claim 4, further comprising a loop or hoop attached to the leash.

6. The valve apparatus of claim 1, wherein the valve apparatus is configured to be attached to the breast pump system.

7. The valve apparatus of claim 2, further comprising a duckbill valve configured in the cylindrical portion.

8. The valve apparatus of claim 2, further comprising an umbrella valve configured in the cylindrical portion.

9. The valve apparatus of claim 2, further comprising a double tailed umbrella valve configured in the cylindrical portion.

10. The valve apparatus of claim 2, further comprising a replaceable component valve structure.

11. The valve apparatus of claim 1, wherein the valve portion includes an opening defining an air path that when occluded facilitates detection of a container assembly.

12. The valve apparatus of claim 1, further comprising a projection extending from the base, the projection defining a non-cylindrical profile.
